(19) United States
(12) Patent Application Publication
(10) Pub. No.: US 2010/0069346 A1
(43) Pub. Date: Mar. 18, 2010

(54) New Azetidine Derivatives as Neurokinin Receptor Antagonists for the Treatment of Gastrointestinal Diseases

(76) Inventors: Sara Holmqvist, Molndal (SE); Anders Chansson, Molndal (SE); Ame Svensson, Molndal (SE); Sverker Von Unge, Molndal (SE)

Correspondence Address: FISH & RICHARDSON P.C., PO BOX 1022, MINNEAPOLIS, MN, 55440-1022, US

(21) Appl. No.: 11/993,361

(22) PCT Filed: Jun. 21, 2006
(86) PCT No.: PCT/SE06/00759

§ 371 Date: Dec. 20, 2007

(30) Foreign Application Priority Data
Jun. 23, 2005 (SE) 0501494-9

(51) Int. Cl. A61K 31/496; C07D 403/04; C07D 413/04; A61P 1/00

(52) U.S. Cl. 514/210.2; 544/359; 544/111

(57) ABSTRACT

The application relates to new piperazine- or morpholine-substituted azetidine derivatives of formula I. These compounds are antagonists at the neurokinin receptor and can be used for the treatment of gastrointestinal diseases. The application also relates to processes for the preparation of the compounds and to intermediates in said preparation.

### FIELD OF THE INVENTION

The present invention relates to new compounds of formula I, to pharmaceutical compositions containing said compounds, and to the use of said compounds in therapy. The present invention further relates to processes for the preparation of compounds of formula I and to new intermediates thereof.

### BACKGROUND OF THE INVENTION

The neurokinins, also known as the tachykinins, comprise a class of peptide neurotransmitters which are found in the peripheral and central nervous systems. The three is principal tachykinins are Substance P (SP), Neurokinin A (NKA) and Neurokinin B (NKB). At least three receptor types are known for the three principal tachykinins. Based upon their relative selectivities favouring the agonists SP, NKA and NKB, the receptors are classified as neurokinin 1 (NK₁), neurokinin 2 (NK₂) and neurokinin 3 (NK₃) receptors, respectively.

There is a need for an orally active NK receptor antagonist for the treatment of e.g. respiratory, cardiovascular, neuro, pain, oncology, inflammatory and/or gastrointestinal disorders. In order to increase the therapeutic index of such therapy it is desirable to obtain such a compound possessing no or minimal toxicity as well as being selective to said NK receptors. Furthermore, it is considered necessary that said medicament has favourable pharmacokinetic and metabolic properties thus providing an improved therapeutic and safety profile such as lower liver enzyme inhibiting properties.

It is well known that severe problems such as toxicity may occur if plasma levels of one medication are altered by the co-administration of another drug. This phenomenon—which is named drug-drug interactions—could happen if there is a change in the metabolism of one drug caused by the co-administration of another substance possessing liver enzyme inhibiting properties. CYP (cytochrome P450) 3A4 is the most important enzyme in the human liver as a majority of oxidised drugs have been biotransformed by this enzyme. Accordingly, it is undesirable to employ a medication having a significant degree of such liver enzyme inhibiting properties. It has been found that many NK receptor antagonists known in the art inhibit the CYP3A4 enzyme to a certain level and consequently there is a possible risk if high doses of those compounds are being used in therapy. Thus, there is a need for a novel NK receptor antagonist with improved pharmacokinetic properties. The present invention provides compounds with CYP3A4 enzyme inhibiting properties at a low level, as comparatively high IC₅₀ values are obtained in a CYP3A4 inhibiting assay. Said method for determining CYP3A4 inhibition is described in Bapiro et al; Drug Metab. Dispos. 29, 30-35 (2001).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current. The aperture-forming alpha sub-units are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Compounds of the present invention have particularly low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

EP 0625509, EP 0630887, WO 95/05377, WO 95/12577, WO 95/15961, WO 96/24582, WO 00/02859, WO 00/20003, WO 00/20389, WO 00/25766, WO 00/34243, WO 02/51807 and WO 03/037889 disclose piperidinylbutylamide derivatives, which are tachykinin antagonists.

“4-Amino-2-(aryl)-butylbenzamides and Their Conformationally Constrained Analogues. Potent Antagonists of the Human Neurokinin-2 (NK₂) Receptor”, Roderick MacKenzie, A., et al, Bioorganic & Medicinal Chemistry Letters (2003), 13, 2211-2215, discloses the compound N-[2-(3,4-dichlorophenyl)-4-(3-morpholin-4-ylazetidin-1-yl)butyl]-N-methylbenzamide which was found to possess functional NK₂ receptor antagonistic properties.

WO 96/05193, WO 97/27185 and EP 0962457 disclose azetidinylalkyllactam derivatives with tachykinin antagonist activity.

EP 0790248 discloses azetidinylalkylazapiperidones and azetidinylalkyloxapiperidones, which are stated to be tachykinin antagonists.

WO 99/01451 and WO 97/25322 disclose azetidinylalkylpiperidine derivatives claimed to be tachykinin antagonists.

EP 0791592 discloses azetidinylalkylglutarimides with tachykinin antagonistic properties.

WO2004/110344 A2 discloses dual NK1,2 antagonists and the use thereof.

An object of the present invention was to provide novel neurokinin antagonists useful in therapy. A further object was to provide novel compounds having improved pharmacokinetic and metabolic properties as well as limited interaction with the hERG channel.

### OUTLINE OF THE INVENTION

The present invention provides a compound of the general formula (I)

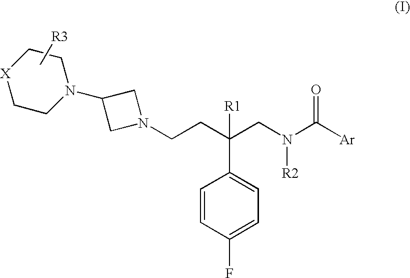

wherein
R1 is hydrogen;
R2 is C₁-C₄ alkyl, wherein one or more of the hydrogen atoms of the allyl group may be substituted for a fluoro atom;
R3 is (CH₂)ₙCR6R7OH; wherein
n is 0, 1, 2 or 3;
X is O or NR4; wherein
R4 is hydrogen, C₁-C₄ alkyl, C₂-C₄ hydroxyalkyl or 2-(dimethylamino)-2-oxoethyl, wherein one or more of the hydrogen atoms of the alkyl group or hydroxyalkyl group may be substituted for a fluoro atom;
R6 is hydrogen or methyl;
R7 is hydrogen or methyl; and
Ar is selected from

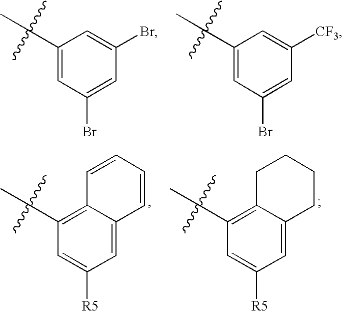

wherein

### to R5 is CN or F;

as well as pharmaceutically and pharmacologically acceptable salts thereof, and

enantiomers of the compound of formula I and salts thereof.

The present invention relates to compounds of formula I as defined above as well as to salts thereof. Salts for use in pharmaceutical compositions will be pharmaceutically acceptable salts, but other salts may be useful in the production of the compounds of formula I.

The compounds of the present invention are capable of forming salts with various inorganic and organic acids and such salts are also within the scope of this invention. Examples of such acid addition salts include acetate, adipate, ascorbate, benzoate, benzenesulfonate, bisulfate, butyrate, camphorate, camphorsulfonate, citrate, cyclohexyl sulfamate, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, 2-naphthalenesulfonate, nitrate, oxalate, palmoate, persulfate, phenylacetate, phosphate, picrate, pivalate, propionate, quinate, salicylate, stearate, succinate, sulfamate, sulfanilate, sulfate, tartrate, tosylate (p-toluenesulfonate), and undecanoate.

Pharmaceutically acceptable salts may be prepared from the corresponding acid in conventional manner. Non-pharmaceutically-acceptable salts may be useful as intermediates and as such are another aspect of the present invention.

Acid addition salts may also be in the form of polymeric salts such as polymeric sulfonates.

The salts may be formed by conventional means, such as by reacting the free base form of the product with one or more equivalents of the appropriate acid in a solvent or medium in which the salt is poorly soluble, or in a solvent such as water, which is removed in vacuo or by freeze drying or by exchanging the anions of an existing salt for another anion on a suitable ion-exchange resin.

Compounds of formula I have one or more chiral centres, and it is to be understood that the invention encompasses all optical isomers, enantiomers and diastereomers. The compounds according to formula (I) can be in the form of the single stereoisomers, i.e. the single enantiomer (the R-enantiomer or the S-enantiomer) and/or diastereomer. The compounds according to formula (I) can also be in the form of a racemic mixture, i.e. an equimolar mixture of enantiomers.

It is to be understood that the present invention also relates to any and all tautomeric forms of the compounds of formula I.

The compounds can exist as a mixture of conformational isomers. The compounds of this invention comprise both mixtures of, and individual, conformational isomers.

Unless stated otherwise, the term “alkyl” includes straight as well as branched chain C₁₋₄ alkyl groups, for example methyl, ethyl, n-propyl, i-propyl, n-butyl, i-butyl, s-butyl or t-butyl. One or more of the hydrogen atoms of the alkyl group may be substituted for a fluoro atom, such as in difluoromethyl or trifluoromethyl.

As used herein, C₁-C₄ hydroxyalkyl is a hydroxyalkyl group comprising 1-4 carbon atoms and a hydroxyl group. One or more of the hydrogen atoms of the hydroxyalkyl group may be substituted for a fluoro atom.

### Pharmaceutical Formulations

According to one aspect of the present invention there is provided a pharmaceutical formulation comprising a compound of formula I, as a single enantiomer, a racemate or a mixture thereof as a free base or pharmaceutically acceptable salts thereof, for use in prevention and/or treatment of respiratory, cardiovascular, neuro, pain, oncology, is inflammatory and/or gastrointestinal disorders.

The pharmaceutical compositions of this invention may be administered in standard manner for the disease condition that it is desired to treat, for example by oral, topical, parenteral, buccal, nasal, vaginal or rectal administration or by inhalation or insufflation. For these purposes the compounds of this invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to herein.

The pharmaceutical compositions of this invention will normally be administered to humans so that, for example, a daily dose of 0.01 to 25 mg/kg body weight (and preferably of 0.1 to 5 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention. For example a tablet or capsule for oral administration may conveniently to contain up to 250 mg (and typically 5 to 100 mg) of a compound of the formula (I) or a pharmaceutically acceptable salt thereof. In another example, for administration by inhalation, a compound of the formula (I) or a pharmaceutically acceptable salt thereof may be administered in a daily dosage range of 5 to 100 mg, in a single dose or divided into two to four daily doses. In a further example, for administration by intravenous or intramuscular injection or infusion, a sterile solution or suspension containing up to 10% w/w (and typically 5% w/w) of a compound of the formula (I) or a pharmaceutically acceptable salt thereof may be used.

### Medical and Pharmaceutical Use

The present invention provides a method of treating or preventing a disease condition wherein antagonism of tachykinins acting at the NK receptors is beneficial which comprises administering to a subject an effective amount of a compound of the formula (I) or a pharmaceutically-acceptable salt thereof. The present invention also provides the use of a compound of the formula (I) or a pharmaceutically acceptable salt thereof in the preparation of a medicament for use in a disease condition wherein antagonism of tachykinins acting at the NK receptors is beneficial.

The compounds of formula (I) or pharmaceutically acceptable salts or solvates thereof may be used in the manufacture of a medicament for use in the prevention or treatment of respiratory, cardiovascular, neuro, pain, oncology and/or gastrointestinal disorders.

Examples of such disorders are asthma, allergic rhinitis, pulmonary diseases, cough, cold, inflammation, chronic obstructive pulmonary disease, airway reactivity, urticaria, hypertension, rheumatoid arthritis, edema, angiogenesis, pain, migraine, tension headache, psychoses, depression, anxiety, Alzheimer's disease, schizophrenia, Huntington's disease, bladder hypermotility, urinary incontinence, eating disorder, manic depression, substance dependence, movement disorder, cognitive disorder, obesity, stress disorders, micturition disorders, mania, hypomania and aggression, bipolar disorder, cancer, carcinoma, fibromyalgia, non cardiac chest pain, gastrointestinal hypermotility, gastric asthma, Crohn's disease, gastric emptying disorders, ulcerative colitis, irritable bowel syndrome (IBS), inflammatory bowel disease (IBD), emesis, gastric asthma, gastric motility disorders, gastro-esophageal reflux disease (GERD) or functional dyspepsia.

### Pharmacology

### Transfection and Culturing of Cells Used in FLIPR and Binding Assays

Chinese Hamster Ovary (CHO) K1 cells (obtained from ATCC) were stably transfected with the human NK₂ receptor (hNK₂R cDNA in pRc/CMV, Invitrogen) or the human NK₃ receptor (hNK₃R in pcDNA 3.1/Hygro (+)/IRES/CD8, Invitrogen vector modified at AstraZeneca EST-Bio UK, Alderley Park). The cells were transfected with the cationic lipid reagent LIPOFECTAMINE™ (Invitrogen) and selection was performed with Geneticin (G418, Invitrogen) at 1 mg/ml for the hNK₂R transfected cells and with Hygromycin (Invitrogen) at 500 μg/ml for the hNK₃R transfected cells. Single cell clones were collected by aid of Fluorescence Activated Cell Sorter (FACS), tested for functionality in a FLIPR assay (see below), expanded in culture and cryopreserved for future use. CHO cells stably transfected with human NK₁ receptors originates from AstraZeneca R&D, Wilmington USA. Human NK₁ receptor cDNA (obtained from RNA-PCR from lung tissue) was subcloned into pRcCMV (Invitrogen). Transfection was performed by Calcium Phosphate and selection with 1 mg/ml G418.

The CHO cells stably transfected with hNK₁R, hNK₂R and hNK₃R were cultured in a humidified incubator under 5% CO₂, in Nut Mix F12 (HAM) with Glutamax I, 10% Foetal Bovine Serum (FBS), 1% Penicillin/Streptomycin (PEST) supplemented with 200 μg/ml Geneticin for the hNK₁R and hNK₂R expressing cells and 500 μg/ml Hygromycin for the hNK₃R expressing cells. The cells were grown in T175 flasks and routinely passaged when 70-80% confluent for up to 20-25 passages.

### Assessing the Activity of Selected Test Compounds to Inhibit Human NK₁/NK₂/NK₃ Receptor Activation (FLIPR Assay)

The activity of a compound of the invention to inhibit NK₁/NK₂/NK₃ receptor activation measured as NK₁/NK₂/NK₃ receptor mediated increase in intracellular Ca²⁺ was assessed by the following procedure:

CHO cells stably transfected with human NK₁, NK₂ or NK₃ receptors were plated in black walled/clear bottomed 96-well plates (Costar 3904) at 3.5×10⁴ cells per well and grown for approximately 24 h in normal growth media in a 37° C. CO₂-incubator.

Before the FLIPR assay the cells of each 96-well plate were loaded with the Ca²⁺ sensitive dye Fluo-3 (TEFLABS 0116) at 4 μM in a loading media consisting of Nut Mix F12 (HAM) with Glutamax I, 22 mM HEPES, 2.5 mM Probenicid (Sigma P-8761) and 0.04% Pluronic F-127 (Sigma P-2443) for 1 h kept dark in a 37° C. CO₂-incubator. The cells were then washed three times in assay buffer (Hanks balanced salt solution (HBSS) containing 20 mM HEPES, 2.5 mM Probenicid and 0.1% BSA) using a multi-channel pipette leaving them in 150 μl at the end of the last wash. Serial dilutions of a test compound in assay buffer (final DMSO concentration kept below 1%) were automatically pipetted by FLIPR (Fluorometric Imaging Plate Reader) into each test well and the fluorescence intensity was recorded (excitation 488 nm and emission 530 nm) by the FLIPR CCD camera for a 2 min pre-incubation period. 50 μl of the Substance P (NK₁ specific), NKA (NK₂ specific), or Pro-7-NKB (NK₃ specific) agonist solution (final concentration equivalent to an approximate EC₆₀ concentration) was then added by FLIPR into each well already containing 200 μl assay buffer (containing the test compound or vehicle) and the fluorescence was continuously monitored for another 2 min. The response was measured as the peak relative fluorescence after agonist addition and IC₅₀s were calculated from ten-point concentration-response curves for each compound. The IC₅₀s were then converted to pK_{B} values with the following formula:

K_{B}=IC₅₀/1+(EC₆₀ conc. of agonist used in assay/EC₅₀ agonist)

pK_{B}=−log K_{B}

### Determining the Dissociation Constant (Ki) of Compounds for Human NK₁/NK₂/NK₃ Receptors (Binding Assay)

Membranes were prepared from CHO cells stably transfected with human NK₁, NK₂ or NK₃ receptors according to the following method.

Cells were detached with Accutase® solution, harvested in PBS containing 5% FBS by centrifugation, washed twice in PBS and resuspended to a concentration of 1×10⁸ cells/ml in Tris-HCl 50 mM, KCl 300 mM, EDTA-N₂ 10 mM pH 7.4 (4° C.). Cell suspensions were is homogenized with an UltraTurrax 30 s 12.000 rpm. The homogenates were centrifuged at 38.000×g (4° C.) and the pellet resuspended in Tris-HCl 50 mM pH 7.4. The homogenization was repeated once and the homogenates were incubated on ice for 45 min. The homogenates were again centrifuged as described above and resuspended in Tris-HCl 50 mM pH 7.4. This centrifugation step was repeated 3 times in total. After the last centrifugation step the pellet was resuspended in Tris-HCl 50 mM and homogenized with Dual Potter, 10 strokes to a homogenous solution, an aliquot was removed for protein determination. Membranes were aliquoted and frozen at −80° C. until use.

The radioligand binding assay is performed at room temperature in 96-well microtiter plates (No-binding Surface Plates, Corning 3600) with a final assay volume of 200 μl/well in incubation buffer (50 mM Tris buffer (pH 7.4 RT) containing 0.1% BSA, 40 mg/L Bacitracin, complete EDTA-free protease inhibitor cocktail tablets 20 pills/L (Roche) and 3 mM MnCl₂). Competition binding curves were done by adding increasing amounts of the test compound. Test compounds were dissolved and serially diluted in DMSO, final DMSO concentration 1.5% in the assay. 50 μl Non labelled ZD 6021 (a non selective NK-antagonist, 10 μM final conc) was added for measurement of non-specific binding. For total binding, 50 μl of 1.5% DMSO (final conc) in incubation buffer was used. [³H-Sar,Met(O₂)-Substance P] (4 nM final cone) was used in binding experiments on hNK₁r. [³H-SR48968] (3 nM final conc.) for hNK₂r and [³H-SR142801] (3 nM final cone) for binding experiments on hNK₃r. 50 μl radioligand, 3 μl test compound diluted in DMSO and 47 μl incubation buffer were mixed with 5-10 μg cell membranes in 100 μl incubation buffer and incubated for 30 min at room temperature on a microplate shaker.

The membranes were then collected by rapid filtration on Filtermat B (Wallac), presoaked in 0.1% BSA and 0.3% Polyethyleneimine (Sigma P-3143), using a Micro 96 Harvester (Skatron Instruments, Norway). Filters were washed by the harvester with ice-cold wash buffer (50 mM Tris-HCl, pH 7.4 at 4° C., containing 3 mM MnCl₂) and dried at 50° C. for 30-60 min. Meltilex scintillator sheets were melted on to filters using a Microsealer (Wallac, Finland) and the filters were counted in a β-Liquid Scintillation Counter (1450 Microbeta, Wallac, Finland).

The Kᵢ value for the unlabeled ligand was calculated using the Cheng-Prusoff equation (Biochem. Pharmacol. 22:3099-3108, 1973): where L is the concentration of the radioactive ligand used and K_{d} is the affinity of the radioactive ligand for the receptor, determined by saturation binding.

Data was fitted to a four-parameter equation using Excel Fit.

Kᵢ=IC₅₀/(1+(L/K_{d}))

### Results

In general, the compounds of the invention, which were tested, demonstrated statistically significant antagonistic activity at the NK₁ receptor within the range of 7-9 for the pK_{B}. For the NK₂ receptor the range for the pK_{B} was 7-9. In general, the antagonistic activity at the NK₃ receptor was 6-9 for the pK_{B}.

In general, the compounds of the invention, which were tested, demonstrated statistically significant CYP3A4 inhibition at a low level. The IC₅₀ values tested according to Bapiro et al; Drug Metab. Dispos. 29, 30-35 (2001) were generally greater than 2 μM.

Activity Against hERG

The activity of compounds according to formula I against the hERG-encoded potassium channel can be determined according to Kiss L, et al. Assay Drug Dev Technol. 1 (2003), 127-35: “High throughput ion-channel pharmacology: planar-array-based voltage clamp”.

In general, the compounds of the invention, which were tested, demonstrated statistically significant hERG activity at a low level. The IC₅₀ values tested as described above were generally greater than 10 μM.

### Metabolic Stability

The metabolic stability of compounds according to formula I can be determined as described below:

The rate of biotransformation can be measured as either metabolite(s) formation or the rate of disappearance of the parent compound. The experimental design involves incubation of low concentrations of substrate (usually 1.0 μM) with liver microsomes (usually 0.5 mg/ml) and taking out aliquots at varying time points (usually 0, 5, 10, 15, 20, 30, 40 min.). The test compound is usually dissolved in DMSO. The DMSO concentration in the incubation mixture is usually 0.1% or less since more solvent can drastically reduce the activities of some CYP450s. Incubations are done in 100 mM potassium phosphate buffer, pH 7.4 and at 37° C. Acetonitrile or methanol is used to stop the reaction.

The following table illustrates the properties of the compounds of the present invention:

### 3-Bromo-N-((2S)-2-(4-fluorophenyl)-4-{3-[(3R)-3-(2-hydroxyethyl)morpholin-4-yl]azetidin-1-yl}butyl)-N-methyl-5-(trifluoromethyl)benzamide (Ex 5)

| pKB | pKB | pKB | IC₅₀ | IC₅₀ | CLint |
|---|---|---|---|---|---|
| (NK1) | (NK2) | (NK3) | (hERG) | (CYP3A4) | (HLM) |
| 8.1 | 7.6 | 7.7 | 18.8 μM | 19.2 μM | 40.7 μL/min/mg |

### Biological Evaluation

### Gerbil Foot Tap (NK1 Specific Test Model)

Male Mongolian gerbils (60-80 g) are purchased from Charles River, Germany. On arrival, they are housed in groups of ten, with food and water ad libitum in temperature and humidity-controlled holding rooms. The animals are allowed at least 7 days to acclimatize to the housing conditions before experiments. Each animal is used only once and euthanized immediately after the experiment by heart punctuation or a lethal overdose of pentobarbital sodium.

Gerbils are anaesthetized with isoflurane. Potential CNS-permeable NK1 receptor antagonists are administered intraperitoneally, intravenously or subcutaneously. The compounds are given at various time points (typically 30-120 minutes) prior to stimulation with agonist.

The gerbils are lightly anaesthetized using isofluorane and a small incision is made in the skin over bregma. 10 pmol of ASMSP, a selective NK1 receptor agonist, is administered icy in a volume of 5 μl using a Hamilton syringe with a needle 4 mm long. The wound is clamped shut and the animal is placed in a small plastic cage and allowed to wake up. The cage is placed on a piece of plastic tubing filled with water and connected to a computer via a pressure transducer. The number of hind feet taps is recorded.

### Fecal Pellet Output (NK2 Specific Test Model)

The in vivo effect (NK2) of the compounds of formula I can be determined by measuring NK2 receptor agonist-induced fecal pellet output using gerbil as described in e.g. The Journal of Pharmacology and Experimental Therapeutics (2001), pp. 559-564.

### Colorectal Distension Model

Colorectal distension (CRD) in gerbils is performed as previously described in rats and mice (Tammpere A, Brusberg M, Axenborg J, Hirsch I, Larsson H, Lindström E. Evaluation of pseudo-affective responses to noxious colorectal distension in rats by manometric recordings. Pain 2005; 116: 220-226; Arvidsson S, Larsson M, Larsson H, Lindström E, Martinez V. Assessment of visceral pain-related pseudo-affective responses to colorectal distension in mice by intracolonic manometric recordings. J Pain 2006; 7: 108-118) with slight modifications. Briefly, gerbils are habituated to Bollmann cages 30-60 min per day for three consecutive days prior to experiments to reduce motion artefacts due to restraint stress. A 2 cm polyethylene balloon (made in-house) with connecting catheter is inserted in the distal colon, 2 cm from the base of the balloon to the anus, during light isoflurane anaesthesia (Forene®, Abbott Scandinavia AB, Solna, Sweden). The catheter is fixed to the tail with tape. The balloons are connected to pressure transducers (P-602, CFM-k33, 100 mmHg, Bronkhorst HI-TEC, Veenendal, The Netherlands). Gerbils are allowed to recover from sedation in the Bollmann cages for at least 15 min before the start of experiments.

A customized barostat (AstraZeneca, Mölndal, Sweden) is used to manage air inflation and balloon pressure control. A customized computer software (PharmLab on-line 4.0) running on a standard computer is used to control the barostat and to perform data collection. The distension paradigm used consists of 12 repeated phasic distensions at 80 mmHg, with a pulse duration of 30 sec at 5 min intervals. Compounds or their respective vehicle are administered as intraperitoneal (i.p.) injections before the CRD paradigm. Each gerbil receives both vehicle and compound on different occasions with at least two days between experiments. Hence, each gerbil serves as its own vehicle control.

The analog input channels are sampled with individual sampling rates, and digital filtering is performed on the signals. The balloon pressure signals are sampled at 50 samples/s. A highpass filter at 1 Hz is used to separate the contraction-induced pressure changes from the slow varying pressure generated by the barostat. A resistance in the airflow between the pressure generator and the pressure transducer further enhances the pressure variations induced by abdominal contractions of the animal. A customized computer software (PharmLab off-line 4.0) is used to quantify the magnitude of highpass-filtered balloon pressure signals. The average rectified value (ARV) of the highpass-filtered balloon pressure signals is calculated for 30 s before the pulse (i.e baseline response) and for the duration of the pulse. When calculating the magnitude of the highpass-filtered balloon pressure signals, the first and last seconds of each pulse are excluded since these reflect artifact signals produced by the barostat during inflation and deflation and do not originate from the animal.

### Methods of Preparation

In another aspect the present invention provides a process for preparing a compound of the formula (I) or salts thereof which process comprises:

a) reacting a compound of the formula (III) with a compound of the formula (IV):

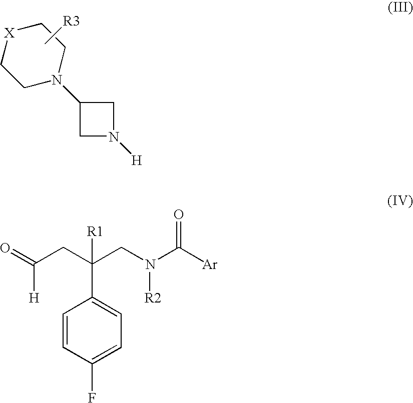

wherein R1-R3 and Ar are as hereinbefore defined; and the conditions are such that reductive alkylation of the compounds of the formula (III) forms an N—C bond between the nitrogen atom of the azetidine group of the compounds of formula (III) and the carbon atom of the aldehyde group of the compounds of formula (IV); or
b) reacting a compound of the formula (III) with a compound of the formula (V):

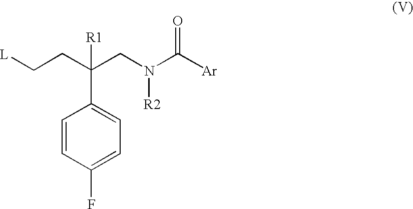

wherein R1-R3 and Ar are as hereinbefore defined; and L is a group such that alkylation of the compounds of the formula (III) forms an N—C bond between the nitrogen atom of the azetidine group of the compounds of formula (III) and the carbon atom of the compounds of formula (V) that is adjacent to the L group; or
c) reacting a compound of the formula (VI) with a compound of the formula (VII):

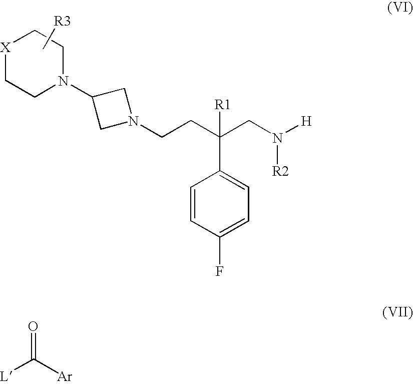

wherein R1-R3 and Ar are as hereinbefore defined; and L′ is a leaving group;
wherein any other functional group is protected, if necessary, and:
i) removing any protecting groups;
ii) optionally oxidizing any oxidizable atoms;
iii) optionally forming a pharmaceutically acceptable salt.

Protecting groups may in general be chosen from any of the groups described in the literature or known to the skilled chemist as appropriate for the protection of the group in question, and may be introduced and removed by conventional methods; see for example Protecting Groups in Organic Chemistry; Theodora W. Greene. Methods of removal are chosen so as to effect removal of the protecting group with minimum disturbance of groups elsewhere in the molecule.

It will also be appreciated that certain of the various optional substituents in the compounds of the formula (I) may be introduced by standard aromatic substitution reactions or generated by conventional functional group modifications either prior to or immediately following the processes described hereinabove. The reagents and reaction conditions for such procedures are well known in the chemical art.

The compounds of the formulae (III) and (IV) are reacted under conditions of reductive alkylation. The reaction is typically performed at a non-extreme temperature, for example 0-40° C., in a substantially inert solvent for example dichloromethane. Typical reducing agents include borohydrides such as sodium cyanoborohydride.

The compounds of the formulae (III) and (V) are reacted under conditions of alkylation. Typically in the compounds of the formula (V) L is a leaving group such as halogen or allylsulfonyloxy. The reaction is typically performed at an elevated temperature, for example 30-130° C., in a substantially inert solvent for example DMF.

The compounds of the formula (III) are known or may be prepared in conventional manner. The compounds of the formula (IV) may be prepared, for example, by reacting a compound of the formula (VII) with a compound of the formula (VIII):

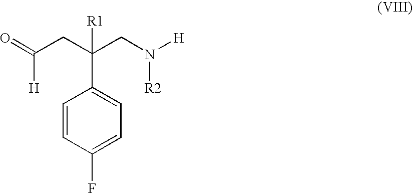

wherein R1-R2 are as hereinbefore defined under conventional acylation conditions.

The compounds of the formula (V) may be prepared, for example, by reacting a compound of the formula (VII) with a compound of the formula (IX):

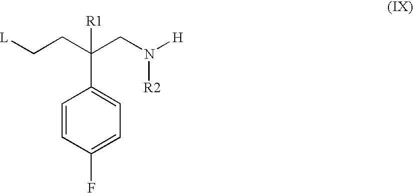

wherein R1-R2 and L are as hereinbefore defined under conventional acylation conditions.

The compounds of the formulae (VI) and (VII) may be reacted under conventional acylation conditions wherein

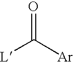

is an acid or an activated acid derivative. Such activated acid derivatives are well known in the literature. They may be formed in situ from the acid or they may be prepared, isolated and subsequently reacted. Typically L′ is chloro thereby forming the acid chloride. Typically the acylation reaction is performed in the presence of a non-nucleophilic base, for example N,N-diisopropylethylamine, in a substantially inert solvent such as dichloromethane at a non-extreme temperature.

The compounds of the formula (VIII) and (IX) are known or may be prepared in conventional manner.

### EXAMPLES

It should be emphasised that the compounds of the present invention most often show highly complex NMR spectra due to the existence of conformational isomers. This is believed to be a result from slow rotation about the amide and/or aryl bond. The following abbreviations are used in the presentation of the NMR data of the compounds: s-singlet; d-doublet; t-triplet; qt-quartet; qn-quintet; m-multiplet; b-broad; cm-complex multiplet, which may include broad peaks.

The following examples will describe, but not limit, the invention.

The following abbreviations are used in the experimental: Boc (tert-butoxycarbonyl), DIPEA (N,N-diisopropylethylamine), DMF (N,N-dimethylformamide), TBTU (N,N,N′,N′-tetramethyl-O-(benzotriazol-1-yl)uronium tetrafluoroborate), THF (tetrahydrofuran) and RT (room temperature).

### Example 1

### 3,5-Dibromo-N-((2S)-2-(4-fluorophenyl)-4-{3-[2-(2-hydroxyethyl)piperazin-1-yl]azetidin-1-yl}butyl)-N-methylbenzamide trihydrochloride

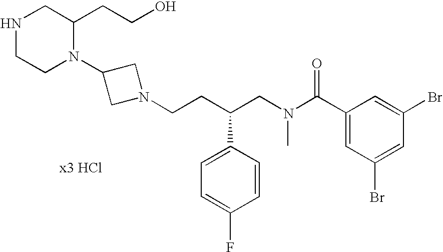

tert-Butyl 4-{1-[(3S)-4-[(3,5-dibromobenzoyl)(methyl)amino]-3-(4-fluorophenyl)butyl]azetidin-3-yl}-3-(2-hydroxyethyl)piperazine-1-carboxylate (see Method 1; 42 mg, 0.058 mmol) was dissolved in a mixture of HCl and dioxane (4M, 10 mL). The solution was stirred at RT for 2 h and then the solvent was removed by evaporation. The residue was dissolved in water and the solution was freeze-dried overnight. There was obtained 45 mg (100%) of the title compound. ¹H NMR (500 MHz, CD₃OD): 0.9-4.4 (cm, 26H), 6.8-7.8 (cm, 7H); LCMS: m/z 627 (M+1)⁺.

### Example 2

### 3-Cyano-N-((2S)-2-(4-fluorophenyl)-4-{3-[2-(2-hydroxyethyl)piperazin-1-yl]azetidin-1-yl}butyl)-N-methyl-5,6,7,8-tetrahydronaphthalene-1-carboxamide trihydrochloride

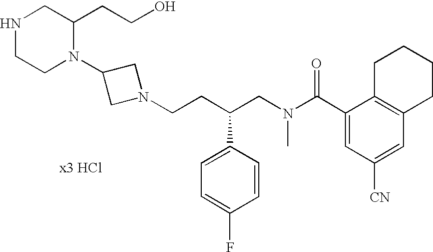

The title compound was prepared by utilizing the acid-catalysed Boc cleavage reaction protocol described in Example 1 but using tert-butyl 4-{1-[(3S)-4-[[(3-cyano-5,6,7,8-tetrahydronaphthalen-1-yl)carbonyl](methyl)amino]-3-(4-fluorophenyl)butyl]azetidin-3-yl}-3-(2-hydroxyethyl)piperazine-1-carboxylate (see Method 2) as the Boc protected amino derivative (yield, 100%). ¹H NMR (500 MHz, CD₃OD): 0.9-4.4 (cm, 26H), 5.7-7.8 (cm, 6H); m/z 658 (M+1)⁺.

### Example 3

### 3-Cyano-N-((2S)-2-(4-fluorophenyl)-4-{3-[2-(hydroxymethyl)piperazin-1-yl]azetidin-1-yl}butyl)-N-methyl-1-naphthamide trihydrochloride

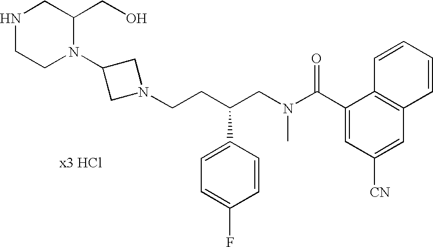

The title compound was prepared by utilizing the acid-catalysed Boc cleavage reaction protocol described in Example 1 but using tert-butyl 4-{1-[(3S)-4-[(3-cyano-1-naphthoyl)(methyl)amino]-3-(4-fluorophenyl)butyl]azetidin-3-yl}-3-(hydroxymethyl)piperazine-1-carboxylate (see Method 3) as the Boc protected amino derivative (yield, 99%). ¹H NMR (500 MHz, CD₃OD): 0.9-4.6 (cm, 24H), 6.2-8.5 (cm, 10H); m/z 630 (M+1)⁺.

### Example 4

### 3,5-Dibromo-N-((2S)-2-(4-fluorophenyl)-4-{3-[2-(hydroxymethyl)piperazin-1-yl]azetidin-1-yl}butyl)-N-methylbenzamide trihydrochloride

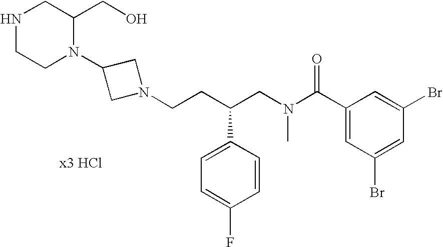

The title compound was prepared by utilizing the acid-catalysed Boc cleavage reaction protocol described in Example 1 but using tert-butyl 4-{1-[(3S)-4-[(3,5-dibromobenzoyl)(methyl)amino]-3-(4-fluorophenyl)butyl]azetidin-3-yl}-3-(hydroxymethyl)piperazine-1-carboxylate (see Method 4) as the Boc protected amino derivative (yield, 99%). ¹H NMR (500 MHz, CD₃OD): 0.9-4.5 (cm, 24H), 6.8-7.8 (cm, 711); m/z 613 (M+1)⁺.

### Example 5

### 3-Bromo-N-((2S)-2-(4-fluorophenyl)-4-{3-[(3R)-3-(2-hydroxyethyl)morpholin-4-yl]azetidin-1-yl}butyl)-N-methyl-5-(trifluoromethyl)benzamide

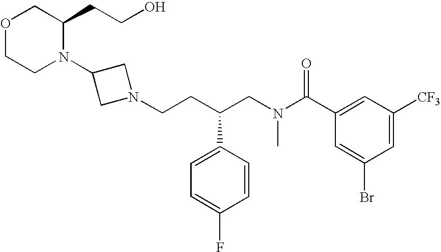

To a mixture of 3-bromo-N-[(2S)-2-(4-fluorophenyl)-4-oxobutyl]-N-methyl-5-(trifluoromethyl)benzamide (see Method 5; 35 mg, 0.078 mmol) and 2-[(3R)-4-azetidin-3-ylmorpholin-3-yl]ethanol (see Method 6; 19 mg, 0.10 mmol) in methanol (2 mL) under nitrogen was added triethylamine (0.03 mL, 0.24 mmol). A mixture of sodium cyano borohydride (34 mg, 0.55 mmol) and zinc chloride (32 mg, 0.24 mmol) in methanol (2 mL) was added and the reaction mixture was stirred at RT overnight. The solvent was removed by evaporation. The residue was dissolved in methylene chloride and the solution was washed twice with aqueous NaHCO₃ and then with brine. The organic phase was separated and the solvent was removed by evaporation. The product was purified by chromatography on silica gel (ammonia saturated methanol-methylene chloride 4 to 8%). There was obtained 30 mg (62%) of the title compound as a white foam. ¹H NMR (500 MHz, CDCl₃): 1.4-1.9 (cm, 4H), 2.2-3.9 (cm, 21H), 6.8-7.4 (cm, 6H), 7.8 (s, 1H); LCMS: m/z 617 (M+1)⁺.

### Example 6

### 3-Cyano-N-((2S)-2-(4-fluorophenyl)-4-{3-[(3R)-3-(2-hydroxyethyl)morpholin-4-yl]azetidin-1-yl}butyl)-N-methyl-5,6,7,8-tetrahydronaphthalene-1-carboxamide

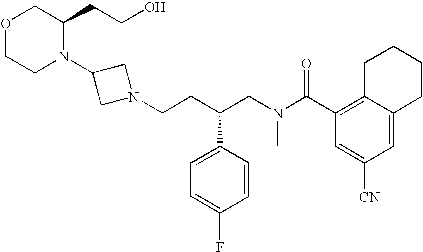

The title compound was prepared by utilizing the reductive amination protocol described in Example 5 but using 3-cyano-N-[(2S)-2-(4-fluorophenyl)-4-oxobutyl]-N-methyl-5,6,7,8-tetrahydronaphthalene-1-carboxamide (see WO 04/110344) as the aldehyde starting material (yield, 52%). ¹H NMR (500 MHz, CDCl₃): 1.3-4.1 (cm, 34H), 6.0-7.4 (cm, 6H); LCMS: m/z 549 (M+1)⁺.

### Example 7

### 3,5-Dibromo-N-((2S)-2-(4-fluorophenyl)-4-{3-[(3R)-3-(2-hydroxyethyl)morpholin-4-yl]azetidin-1-yl}butyl)-N-methylbenzamide

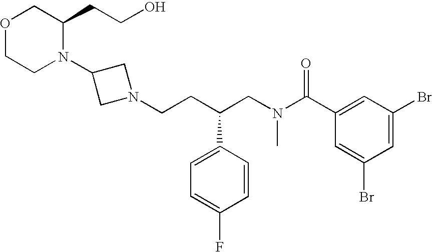

The title compound was prepared by utilizing the reductive amination protocol described in Example 5 but using 3,5-dibromo-N-[(2S)-2-(4-fluorophenyl)-4-oxobutyl]-N-methylbenzamide (see WO 04/110344) as the aldehyde starting material (yield, 41%). ¹H NMR (500 MHz, CDCl₃): 1.3-3.8 (cm, 26H), 6.8-7.3 (cm, 6H), 7.8 (s, 1H); LCMS: m/z 628 (M+1)⁺.

### Example 8

### 3,5-Dibromo-N-((2S)-2-(4-fluorophenyl)-4-{3-[(3R)-3-hydroxymethyl)morpholin-4-yl]azetidin-1-yl}butyl)-N-methylbenzamide

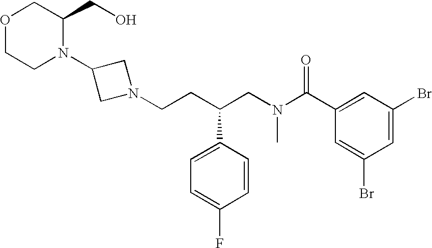

The title compound was prepared by utilizing the reductive amination protocol described in Example 5 but using 3,5-dibromo-N-[(2S)-2-(4-fluorophenyl)-4-oxobutyl]-N-methylbenzamide (see WO 04/110344) as the aldehyde starting material and [(3R)-4-azetidin-3-ylmorpholin-3-yl]methanol (see Method 7) as the azetidine starting material (yield, 22%). ¹H NMR (500 MHz, CDCl₃): 1.3-3.8 (cm, 24H), 6.8-7.4 (cm, 6H), 7.9 (s, 1H); LCMS: m/z 614 (M+1)⁺.

### Example 9

### 3-Cyano-N-((2S)-2-(4-fluorophenyl)-4-{3-[(3R)-3-(hydroxymethyl)morpholin-4-yl]azetidin-1-yl}butyl)-N-methyl-5,6,7,8-tetrahydronaphthalene-1-carboxamide

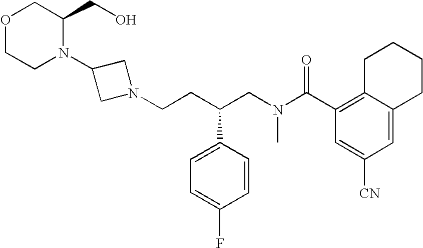

The title compound was prepared by utilizing the reductive amination protocol described in Example 5 but using 3-cyano-N-[(2S)-2-(4-fluorophenyl)-4-oxobutyl]-N-methyl-5,6,7,8-tetrahydronaphthalene-1-carboxamide (see WO 04/110344) as the aldehyde starting material and [(3R)-4-azetidin-3-ylmorpholin-3-yl]methanol (see Method 7) as the azetidine starting material (yield, 79%). ¹H NMR (500 MHz, CDCl₃): 1.3-4.1 (cm, 32H), 6.0-7.4 (cm, 6H); LCMS: m/z 535 (M+1)⁺.

### Preparation of Starting Materials

The starting materials for the examples above are either commercially available or are readily prepared by standard methods from known materials. For example, the following reactions are an illustration, but not a limitation, of some of the starting materials.

### Method 1

### tert-Butyl 4-{1-[(3S)-4-[(3,5-dibromobenzoyl)(methyl)amino]-3-(4-fluorophenyl)butyl]azetidin-3-yl}-3-(2-hydroxyethyl)piperazine-1-carboxylate

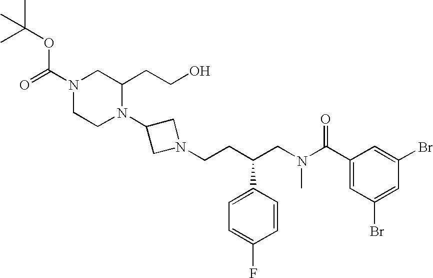

### (a) Ethyl {1-[1-(diphenylmethyl)azetidin-3-yl]-3-oxopiperazin-2-yl}acetate

A solution of 1-(diphenylmethyl)azetidin-3-yl methanesulfonate (see J. Org. Chem.; 56; 1991; 6729; 1.97 g, 6.2 mmol), ethyl 2-(3-oxo-2-piperazinyl)acetate (1.34 g, 3.78 mmol), triethylamine (1.0 mL, 7.2 mmol) and acetonitrile (100 mL) was heated to reflux for 5 days. The solvent was removed by evaporation and the residue was dissolved in methylene chloride. The solution was washed with aqueous NaHCO₃. The organic solution was separated and the solvent was removed by evaporation. The product was purified by chromatography on silica gel (methanol-methylene chloride 5:95). There was obtained 0.86 g (34%) of ethyl {1-[1-(diphenylmethyl)azetidin-3-yl]-3-oxopiperazin-2-yl}acetate as an oil. ¹H NMR (500 MHz, CDCl₃): 1.2 (t, 3H), 2.6 (m, 1H), 2.7-2.8 (m, 2H), 2.9 (q, 2H), 2.9-3.0 (m, 1H), 3.2 (m, 1H), 3.3-3.4 (m, 2H), 3.4-3.5 (m, 2H), 3.5-3.6 (m, 1H), 4.1-4.2 (m, 2H), 4.3 (s, 1H), 7.2 (t, 2H), 7.3 (t, 4H), 7.4 (d, 4H).

### (b) 2-{1-[1-(Diphenylmethyl)azetidin-3-yl]piperazin-2-yl}ethanol

To an ice-cooled suspension of LiAlH₄ (0.33 g, 8.7 mmol) in THF (10 mL) under nitrogen was added ethyl {1-[1-(diphenylmethyl)azetidin-3-yl]-3-oxopiperazin-2-yl}acetate (0.86 g, 2.1 mmol) dissolved in THF (10 mL). The mixture was stirred at 0° C. for 2 h and then three teaspoonfuls of Na₂SO₄×10H₂O was added. The mixture was filtered through Celite® and the filter cake was washed with THF and then with water. The solvent was removed by evaporation and to the residue was added a saturated aqueous solution of NaHCO₃. The solution was extracted twice with methylene chloride. The organic solution was separated and the solvent was removed by evaporation. A more close analysis of the crude product (0.51 g) showed that all material had not been reduced completely and thus the above procedure was repeated once more using additional LiAlH₄ (0.25 g, 6.6 mmol). There was obtained 0.40 g (54%) of 2-{1-[1-(diphenylmethyl)azetidin-3-yl]piperazin-2-yl}ethanol as an oil. ¹H NMR (500 MHz, CDCl₃): 1.6 (m, 1H), 1.9-2.0 (m, 1H), 2.2 (m, 1H), 2.6 (m, 1H), 2.7 (dd, 1H), 2.7-2.9 (m, 5H), 3.0 (dd, 1H), 3.4 (m, 1H), 3.5 (m, 1H), 3.6 (m, 1H), 3.7 (m, 2H), 4.4 (s, 1H), 7.2 (t, 2H), 7.3 (m, 4H), 7.4 (m, 4H).

### (c) tert-Butyl 4-[1-(diphenylmethyl)azetidin-3-yl]-3-(2-hydroxyethyl)piperazine-1-carboxylate

To a mixture of 2-{1-[1-(diphenylmethyl)azetidin-3-yl]piperazin-2-yl}ethanol (0.40 g, 1.1 mmol), di-tert-butyldicarbonate (0.24 g, 1.1 mmol) and methylene chloride (50 mL) was added triethylamine (0.16 mL, 1.1 mmol). The mixture was stirred at RT for 20 h and then the solvent was removed by evaporation. The residue was dissolved in ethyl acetate and the solution was extracted with aqueous HCl (0.1 M). The aqueous solution was separated, neutralized by the addition of NaHCO₃ and then extracted with ethyl acetate. The organic phase was dried over MgSO₄ and the solvent was removed by evaporation. The product was purified by chromatography on silica gel (methylene chloride). There was obtained 0.23 g (45%) of tert-butyl 4-[1-(diphenylmethyl)azetidin-3-yl]-3-(2-hydroxyethyl)-piperazine-1-carboxylate. ¹H NMR (500 MHz, CDCl₃): 1.4 (s, 9H), 1.6-1.8 (b, 1H), 1.9 (m, 1H), 2.3 (b, 1H), 2.6 (m, 1H), 2.8 (t, 2H), 2.9 (t, 1H), 3.1-3.2 (b, 1H), 3.3 (dd, 1H), 3.4 (m, 1H), 3.5-3.8 (m, 6H), 4.4 (s, 1H), 7.2 (t, 2H), 7.3 (m, 4H), 7.4 (m, 4H).

### (d) tert-Butyl 4-azetidin-3-yl-3-(2-hydroxyethyl)piperazine-1-carboxylate

A reaction vessel was loaded with palladium hydroxide (20% on carbon, 150 mg) and a solution of tert-butyl 4-[1-(diphenylmethyl)azetidin-3-yl]-3-(2-hydroxyethyl)-piperazine-1-carboxylate (0.23 g, 0.51 mmol) in acetic acid (15 mL). The mixture was stirred under hydrogen for 24 h at 5 bar and RT. The catalyst was filtered off and the filtrate was concentrated. The product was purified on a cation exchange column (Isolute SCX-2). The column was first washed with ethanol and then the product was eluted with ammonia-saturated methanol. The solvent was removed by evaporation and there was obtained 0.15 g (100%) of tert-butyl 4-azetidin-3-yl-3-(2-hydroxyethyl)piperazine-1-carboxylate. ¹H NMR (500 MHz, CDCl₃): 1.4 (s, 9H), 1.5 (m, 1H), 1.7 (m, 1H), 2.3 (b, 1H), 2.6-3.8 (m, 13H).

### (e) tert-Butyl 4-{1-[(3S)-4-[(3,5-dibromobenzoyl)(methyl)amino]-3-(4-fluorophenyl)butyl]azetidin-3-yl}-3-(2-hydroxyethyl)piperazine-1-carboxylate

To a solution of tert-butyl 4-azetidin-3-yl-3-(2-hydroxyethyl)piperazine-1-carboxylate (43 mg, 0.15 mmol) and 3,5-dibromo-N-[(2S)-2-(4-fluorophenyl)-4-oxobutyl]-N-methylbenzamide (see WO 04/110344; 85 mg, 0.19 mmol) in methanol (15 mL) was added a mixture of sodium cyano borohydride (65 mg, 1.0 mmol), zinc chloride (150 mg, 1.1 to mmol) and methanol (5 mL). The reaction mixture was stirred at RT for 30 min and then the solvent was removed by evaporation. The residue was dissolved in ethyl acetate and the solution was washed with aqueous NaHCO₃. The organic phase was separated and then the solvent was removed by evaporation. The product was purified by means of reversed phase chromatography using a mixture of acetonitrile and aqueous 0.1 M ammonium acetate. There was obtained 42 mg (38%) of the title compound as an oil. LCMS: m/z 727 (M+1)⁺.

### Method 2

### tert-Butyl 4-{1-[(3S)-4-[[(3-cyano-5,6,7,8-tetrahydronaphthalen-1-yl)carbonyl](methyl)amino]-3-(4-fluorophenyl)butyl]azetidin-3-yl}-3-(2-hydroxyethyl)piperazine-1-carboxylate

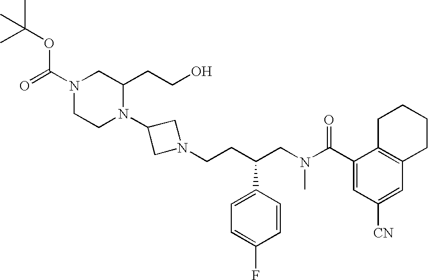

The title compound was prepared by utilizing the reductive amination protocol described in Method 1e but using 3-cyano-N-[(2S)-2-(4-fluorophenyl)-4-oxobutyl]-N-methyl-5,6,7,8-tetrahydronaphthalene-1-carboxamide (see WO 04/110344) as the aldehyde starting material (yield, 24%). ¹H NMR (500 MHz, CDCl₃): 1.4 (s, 9H), 1.5-4.2 (cm, 34H), 6.0-7.4 (cm, 6H).

### Method 3

### tert-Butyl 4-{1-[(3S)-4-[(3-cyano-1-naphthoyl)(methyl)amino]-3-(4-fluorophenyl)butyl]azetidin-3-yl}-3-(hydroxymethyl)piperazine-1-carboxylate

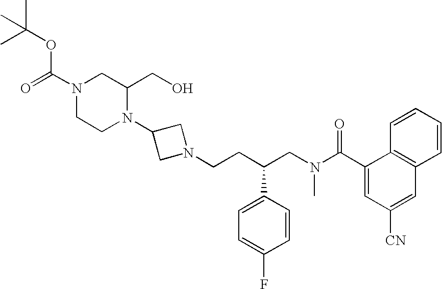

The title compound was prepared by utilizing the reductive amination protocol described in Method 1e but using 3-cyano-N-[(2S)-2-(4-fluorophenyl)-4-oxobutyl]-N-methyl-1-naphthamide (see WO 04/110344) as the aldehyde starting material and tert-butyl 4-azetidin-3-yl-3-(hydroxymethyl)piperazine-1-carboxylate (see Method 7) as the azetidine starting material (yield, 24%). ¹H NMR (500 MHz, CD₃OD): 1.4 (s, 9H), 1.7-4.4 (cm, 24H), 6.3-8.1 (cm, 9H), 8.4 (s, 1H); LCMS: m/z 630 (M+1)⁺.

### Method 4

### tert-Butyl 4-{1-[(3S)-4-[(3,5-dibromobenzoyl)(methyl)amino]-3-(4-fluorophenyl)butyl]azetidin-3-yl}-3-(hydroxymethyl)piperazine-1-carboxylate

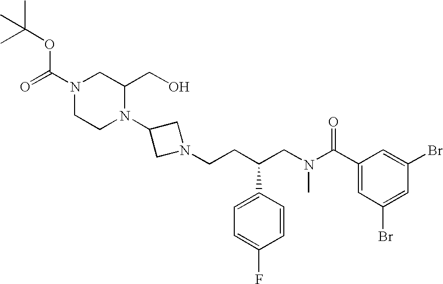

The title compound was prepared by utilizing the reductive amination protocol described in Method 1e but using tert-butyl 4-azetidin-3-yl-3-(hydroxymethyl)piperazine-1-carboxylate (see Method 7) as the azetidine starting material (yield, 27%). ¹H NMR (500 MHz, CD₃OD): 1.4 (s, 9H), 1.6-3.8 (cm, 24H), 6.3-8.1 (cm, 9H), 6.9-7.1 (cm, 3H), 7.1 (t, 1H), 7.2 (s, 1H), 7.3 (t, 1H), 7.8 (d, 1H); LCMS: m/z 713 (M+1)⁺.

### Method 5

### 3-Bromo-N-[(2S)-2-(4-fluorophenyl)-4-oxobutyl]-N-methyl-5-(trifluoromethyl)benzamide

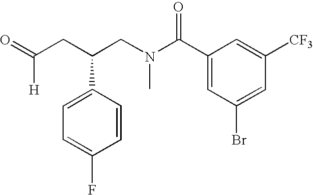

### (a) 3-Bromo-N-[(2S)-2-(4-fluorophenyl)pent-4-en-1-yl]-N-methyl-5-(trifluoromethyl)benzamide

To a solution of [(2S)-2-(4-fluorophenyl)pent-4-en-1-yl]methylamine (see Bioorg. Med. Chem. Lett; 2001; 265-270; 0.54 g, 2.8 mmol) and 3-bromo-5-trifluoromethyl benzoic acid (0.81 g, 3.0 mmol) in DMF (7 mL) was added TBTU (0.96 g, 3.0 mmol) and DIPEA (1.41 g, 10.9 mmol). The reaction mixture was stirred under nitrogen overnight at RT and then partitioned between ethyl acetate and an aqueous NaHCO₃ solution. The aqueous phase was extracted trice with ethyl acetate. The combined organic solutions were washed trice with water and then dried by a phase separator column. The solvent was removed by evaporation and the product was purified by chromatography on silica gel (ethyl acetate-heptane 10% to 17%). There was obtained 0.86 g (68%) of 3-bromo-N-[(2S)-2-(4-fluorophenyl)pent-4-en-1-yl]-N-methyl-5-(trifluoromethyl)benzamide. ¹H NMR (500 MHz, CDCl₃): 2.1-3.8 (cm, 8H), 4.9-5.1 (m, 2H), 5.5-5.8 (m, 1H), 6.8-7.4 (cm, 6H), 7.8 (s, 1H). LCMS: m/z 445 (M+1)⁺.

### (b) 3-Bromo-N-[(2S)-2-(4-fluorophenyl)-4-oxobutyl]-N-methyl-5-(trifluoromethyl)benzamide

To a solution of 3-bromo-N-[(2S)-2-(4-fluorophenyl)pent-4-en-1-yl]-N-methyl-5-(trifluoromethyl)benzamide (0.86 g, 1.9 mmol) in acetone (45 mL) were added OsO₄ (2.5% in t-butyl alcohol, 0.49 ml, 0.039 mmol) and 4-methylmorpholine-4-oxide (0.41 g, 3.5 mmol). The solution was stirred under nitrogen at RT overnight and then an aqueous solution of NaHSO₃ (39%, 45 mL) was added. The mixture was stirred for 2 h, diluted with water and then extracted twice with methylene chloride. The combined organic solutions were separated by means of a phase separator column and the solvent was removed by evaporation. The residue (1.08 g) was dissolved in THF (18 mL) and water (4.5 mL) and to the resultant solution was added NaIO₄ (0.73 g, 3.4 mmol). The mixture was stirred under nitrogen overnight at RT. The mixture was partitioned between methylene chloride and water. The aqueous phase was extracted with methylene chloride and then the combined organic solutions were washed with brine and separated by means of a phase separator column. The solvent was removed by evaporation and there was obtained 0.78 g (90%) of the title compound. ¹H NMR (500 MHz, CDCl₃): 2.4-4.4 (cm, 8H), 6.8-7.8 (cm, 7H), 9.8 (s, 1H); LCMS: m/z 447 (M+1)⁺.

### Method 6

### 2-[(3R)-4-Azetidin-3-ylmorpholin-3-yl]ethanol

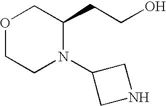

### (a) (3S)-4-Benzyl-3-(chloromethyl)morpholine

To a solution of [(3R)-4-benzylmorpholin-3-yl]methanol (see J. Med. Chem.; 29; 1986; 1288-1290; 1.83 g, 8.8 mmol) in dry methylene chloride (15 mL) was added thionyl chloride (3.15 g, 26.5 mmol) and DMF (2 drops). The mixture was heated to reflux for 2 h 30 min and then the solvent was removed by evaporation. The residue was treated with aqueous NaHCO₃ and the solution was extracted with ethyl acetate. The organic solution was separated and the solvent was removed by evaporation. There was obtained 1.88 g (94%) of (3S)-4-benzyl-3-(chloromethyl)morpholine as an oil. ¹H NMR (500 MHz, CDCl₃): 2.3-2.4 (m, 1H), 2.7 (m, 1H), 2.8 (m, 1H), 3.5 (d, 1H), 3.6-3.9 (m, 5H), 4.0 (d, 1H), 7.3 (m, 1H), 7.4 (m, 4H); LCMS: m/z 226 (M+1)⁺.

### (b) (3R)-4-Benzylmorpholine-3-carbonitrile

To a solution of (3S)-4-benzyl-3-(chloromethyl)morpholine (1.83 g, 8.1 mmol) in methylene chloride (6 mL) was added a mixture of tetrabutylammonium hydrogensulfate (0.14 g, 0.42 mmol), NaOH (0.033 g, 0.83 mmol) and water (6 mL) followed by KCN (0.54 g, 8.3 mmol). The mixture was refluxed for 20 h and then diluted with methylene chloride. The organic phase was washed twice with water and then separated by means of a phase separator column. The solvent was removed by evaporation and the product was purified by chromatography on silica gel (methanol-methylene chloride 0 to 5%). There was obtained 1.66 g (95%) of (3R)-4-benzylmorpholine-3-carbonitrile. ¹H NMR (500 MHz, CDCl₃): 2.4 (m, 1H), 2.6 (dd, 1H), 2.6-2.7 (m, 1H), 2.8 (dd, 1H), 2.9 (m, 1H), 3.4 (d, 1H), 3.7-3.9 (m, 5H), 7.3 (m, 1H), 7.4 (m, 4H): m/z 217 (M+1)⁺.

### (c) Methyl [(3R)-4-benzylmorpholin-3-yl]acetate

(3R)-4-Benzylmorpholine-3-carbonitrile (0.50 g, 2.3 mmol) was dissolved in an HCl-saturated solution of methanol (10 mL). The mixture was stirred at RT overnight and then diluted with water (10 mL). After 10 min at RT most of the methanol was removed by evaporation. The aqueous solution was neutralized by the addition of Na₂CO₃ and then extracted twice with ethyl acetate. The organic solution was separated by means of a phase separator column and then the solvent was removed by evaporation. There was obtained 0.52 g (90%) of methyl [(3R)-4-benzylmorpholin-3-yl]acetate. ¹H NMR (500 MHz, CDCl₃): 2.2 (m, 1H), 2.5-2.6 (m, 3H), 3.0 (m, 1H), 3.3 (d, 1H), 3.5-3.8 (m, 8H), 7.2-7.3 (m, 5H).

### (d) 2-[(3R)-4-Benzylmorpholin-3-yl]ethanol

To a suspension of LiAlH₄ (0.79 g, 20.8 mmol) in ether (3 mL) was added methyl [(3R)-4-benzylmorpholin-3-yl]acetate (0.52 g, 2.1 mmol) dissolved in ether (2 mL). The mixture was stirred at RT for 1 h and then cooled to 0° C. Excess of LiAlH₄ was decomposed by the successive addition of ethyl acetate and saturated aqueous NaHCO₃. To the mixture was added KH₂PO₄ (4 g) and the mixture was then dried by the addition of anhydrous Na₂SO₄. Insoluble material was removed by filtration and the solvent was removed by evaporation. There was obtained 0.42 g (92%) of 2-[(3R)-4-benzylmorpholin-3-yl]ethanol. ¹H NMR (500 MHz, CDCl₃): 1.8 (m, 1H), 2.1 (m, 1H), 2.2 (m, 1H), 2.7 (m, 1H), 2.8 (m, 1H), 3.3 (d, 1H), 3.5-4.0 (m, 7H), 4.3 (d, 1H), 7.2-7.4 (m, 5H).

### (e) 2-[(3R)-Morpholin-3-yl]ethanol

To a solution of 2-[(3R)-4-benzylmorpholin-3-yl]ethanol (0.42 g, 1.9 mmol) in ethanol (10 mL) was added palladium hydroxide (20% on carbon, 0.27 g) and acetic acid (0.2 mL). The mixture was stirred under hydrogen at 4 bar and RT for 17 h. The catalyst was filtered off and the solvent was removed by evaporation. The residue was re-dissolved in ethanol (1 mL) and THF (10 mL). The solution was filtered through a cation exchange column (Isolute SCX-2, 10 g). The column was washed with THF and then the product was eluted with ammonia-saturated methanol. The solvent was removed by evaporation and there was obtained 0.24 g (98%) of 2-[(3R)-morpholin-3-yl]ethanol as an oil. ¹H NMR (500 MHz, CD₃OD): 1.5-1.6 (m, 2H), 2.8-3.0 (m, 3H), 3.2 (t, 1H), 3.5 (m, 1H), 3.6-3.7 (t, 2H), 3.8 (m, 2H).

### (f) 2-{(3R)-4-[7-(Diphenylmethyl)azetidin-3-yl]morpholin-3-yl}ethanol

To a solution of 2-[(3R)-morpholin-3-yl]ethanol (0.24 g, 1.9 mmol) and 1-(diphenylmethyl)azetidin-3-one (see Bioorg. Med. Chem. Lett.; 13; 2003; 2191-2194; 0.42 g, 1.8 mmol) in methanol (5.5 mL) was added acetic acid (0.6 mL). The solution was mixed with (polystyrylmethyl) trimethylammonium cyanoborohydride (4.2 mmol/g, 0.46 g, 2.4 mmol). The mixture was heated for 5 min at 120° C. using microwave single node heating. The solution was filtered and then the solvent was removed by evaporation. The residue was dissolved in methylene chloride and the solution was washed with aqueous NaHCO₃ solution. The organic solution was separated by use of a phase separator column. The solvent was removed by evaporation and the product was chromatographed on silica gel (methylene chloride-ammonia saturated methanol 94:4). There was obtained 0.33 g (50%) of 2-{(3R)-4-[1-(diphenylmethyl)azetidin-3-yl]morpholin-3-yl}ethanol. ¹H NMR (500 MHz, CDCl₃): 1.6 (m, 1H), 1.9 (m, 1H), 2.2 (m, 1H), 2.5 (m, 1H), 2.7-3.0 (m, 3H), 3.4-3.8 (m, 9H), 4.4 (s, 1H), 7.2 (m, 2H), 7.3 (m, 4H), 7.4 (m, 41-1); LCMS: m/z 353 (M+1)⁺.

### (g) 2-[(3R)-4-Azetidin-3-ylmorpholin-3-yl]ethanol

A solution of 2-{(3R)-4-[1-(diphenylmethyl)azetidin-3-yl]morpholin-3-yl}ethanol (0.33 g, 0.93 mmol) in ethanol (8 mL) was mixed with palladium hydroxide (20% on carbon, 0.13 g) and a catalytic amount of acetic acid. The mixture was stirred under hydrogen overnight at 5 bar and RT. The catalyst was filtered off and the solvent was removed by evaporation. The residue was re-dissolved in ethanol (1 mL) and THF (10 mL). The solution was filtered through a strong cation exchange column (Isolute SCX-2, 10 g). The column was washed with TIT and then the product was eluted with ammonia-saturated methanol. The solvent was removed by evaporation and there was obtained 0.17 g (97%) of the title compound as an oil. ¹H NMR (500 MHz, CD₃OD): 1.6-1.8 (b, 2H), 2.3-2.8 (m, 3H), 3.3-4.1 (m, 6H); LCMS: m/z 187 (M+1)⁺.

### Method 7

### [(3R)-4-Azetidin-3-ylmorpholin-3-yl]methanol

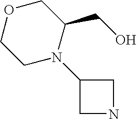

### (a) (3R)-Morpholin-3-ylmethanol

A solution of [(3R)-4-benzylmorpholin-3-yl]methanol (see J. Med. Chem.; 29; 1986; 1288-1290; 1.1 g, 5.4 mmol) in ethanol (25 mL) was mixed with palladium hydroxide (20% on carbon, 0.7 g) and acetic acid (0.5 mL). The mixture was stirred under hydrogen overnight at 1.2 bar and RT. The catalyst was filtered off and the solvent was removed by evaporation. The residue (except 200 mg) was dissolved in ether (1 mL) and THF (10 mL). The solution was filtered through a strong cation exchange column (Isolute SCX-2, 10 g). The column was washed with THF and then the product was eluted with ammonia-saturated methanol. The solvent was removed by evaporation and there was obtained 0.36 g (57%) of (3R)-morpholin-3-ylmethanol as an oil. ¹H NMR (500 MHz, CD₃OD): 2.9 (m, 3H), 3.3 (t, 1H), 3.5 (m, 3H), 3.7-3.9 (m, 2H).

### (b) {(3R)-4-[1-(Diphenylmethyl)azetidin-3-yl]morpholin-3-yl}methanol

A solution of 1-(diphenylmethyl)azetidin-3-yl methanesulfonate (0.32 g, 1.0 mmol), (3R)-morpholin-3-ylmethanol (0.12 g, 1.0 mmol), DIPEA (0.52 mL, 3.0 mmol) and acetonitrile (2.5 mL) was heated at 150° C. using microwave single node heating. The solvent was removed by evaporation. The residue was dissolved in methylene chloride and the solution was washed twice with aqueous NaHCO₃. The organic solution was dried using a phase separator column and then the solvent was removed by evaporation. The product was purified by chromatography on silica gel (methylene chloride-ammonia saturated methanol 1% to 4%). There was obtained 0.17 g (51%) of {(3R)-4-[1-(diphenylmethyl)-azetidin-3-yl]morpholin-3-yl}methanol. ¹H NMR (500 MHz, CDCl₃): 2.2 (m, 1H), 2.3 (m, 1H), 2.8-3.0 (m, 4H), 3.3-3.6 (m, 6H), 3.7-3.8 (m, 2H), 4.4 (s, 1H), 7.2 (m, 2H), 7.3 (m, 4H), 7.4 (m, 4H); LCMS: m/z 339 (M+1)⁺.

### (c) [(3R)-4-Azetidin-3-ylmorpholin-3-yl]methanol

A solution of {(3R)-4-[1-(diphenylmethyl)-azetidin-3-yl]morpholin-3-yl}methanol (0.25 g, 0.74 mmol) in ethanol (6 mL) was mixed with palladium hydroxide (20% on carbon, 0.10 g) and a catalytic amount of acetic acid. The mixture was stirred under hydrogen overnight at 5 bar and RT. The catalyst was filtered off and the solvent was removed by evaporation. The residue was dissolved in methanol (1 mL) and THY (10 mL). The solution was filtered through a strong cation exchange column (Isolute SCX-2, 10 g). The column was washed with THY and then the product was eluted with ammonia-saturated methanol. The solvent was removed by evaporation and there was obtained 0.17 g (66%) of the title compound as an oil. ¹H NMR (500 MHz, CD₃OD): 2.3 (m, 1H), 2.5 (m, 1H), 2.7 (m, 1H), 3.7-3.9 (m, 9H); LCMS: m/z 173 (M+1)⁺.

1. A compound of formula (I) 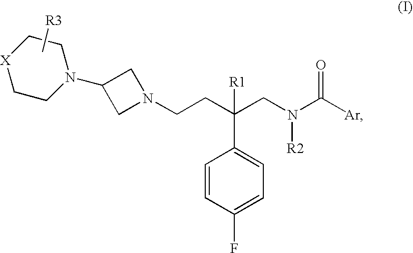
an enantiomer thereof or a pharmaceutically acceptable salt of the compound or enantiomer,
wherein
R1 is hydrogen;
R2 is C₁-C₄ alkyl, wherein the the alkyl group may be substituted with one or more fluoro atoms;
R3 is (CH₂)ₙCR6R7OH; wherein
n is 0, 1, 2 or 3;
X is O or NR4; wherein
R4 is hydrogen, C₁-C₄ alkyl, C₂-C₄ hydroxyalkyl or 2-(dimethylamino)-2-oxoethyl, wherein the alkyl group or hydroxyalkyl group may be substituted with one or more fluoro atoms;
R6 is hydrogen or methyl;
R7 is hydrogen or methyl; and
Ar is selected from 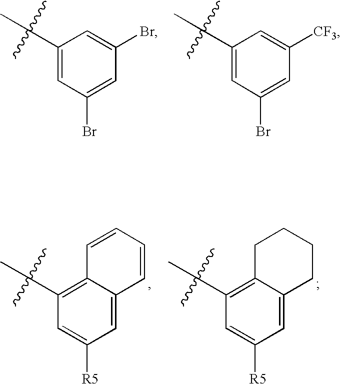
wherein
R5 is CN or F.

2. The compound according to claim 1 wherein Ar is selected from

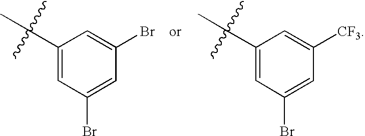

3. The compound according to claim 1 wherein Ar is selected from 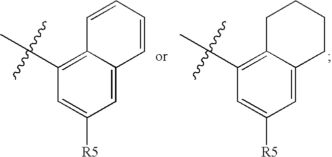
wherein R5 is CN or F.

4. The compound according to any one of claims 1-3 wherein R2 is methyl, wherein the methyl group may be substituted with one or more fluoro atoms.

5. The compound according to any one of claims 1-3 wherein R6 is hydrogen.

6. The compound according to any one of claims 1-3 wherein R7 is hydrogen.

7. The compound according to any one of claims 1-3 wherein R6 is methyl.

8. The compound according to claim 7 wherein R7 is methyl.

9. The compound according to any one of claims 1-3 wherein n is 1 or 2.

10. The compound according to any one of claims 1-3 wherein X is O.

11. The compound according to any one of claims 1-3 wherein X is NR4.

12. The compound according to claim 11 wherein R4 is hydrogen or C₁-C₂ alkyl, wherein the alkyl group may be substituted with one or more fluoro atoms.

13. The compound according to any one of claims 1-3 wherein the compound is the (S)-enantiomer.

14. The compound according to claim 1 selected from
3,5-Dibromo-N-((2S)-2-(4-fluorophenyl)-4-{3-[2-(2-hydroxyethyl)piperazin-1-yl]azetidin-1-yl}butyl)-N-methylbenzamide trihydrochloride;
3-Cyano-N-((2S)-2-(4-fluorophenyl)-4-{3-[2-hydroxyethyl)piperazin-1-yl]azetidin-1-yl}butyl)-N-methyl-5,6,7,8-tetrahydronaphthalene-1-carboxamide trihydrochloride;
3-Cyano-N-((2S)-2-(4-fluorophenyl)-4-{3-[2-(hydroxymethyl)piperazin-1-yl]azetidin-1-yl}butyl)-N-methyl-1-naphthamide trihydrochloride;
3,5-Dibromo-N-((2S)-2-(4-fluorophenyl)-4-{3-[2-(hydroxymethyl)piperazin-1-yl]azetidin-1-yl}butyl)-N-methylbenzamide;
3-Bromo-N-((2S)-2-(4-fluorophenyl)-4-{3-[(3R)-3-(2-hydroxyethyl)morpholin-4-yl]azetidin-1-yl}butyl)-N-methyl-5-(trifluoromethyl)benzamide;
3-Cyano-N-((2S)-2-(4-fluorophenyl)-4-{3-[(3R)-3-(2-hydroxyethyl)morpholin-4-yl]azetidin-1-yl}butyl)-N-methyl-5,6,7,8-tetrahydronaphthalene-1-carboxamide;
3,5-Dibromo-N-((2S)-2-(4-fluorophenyl)-4-{3-[(3R)-3-(2-hydroxyethyl)morpholin-4-yl]azetidin-1-yl}butyl)-N-methylbenzamide;
3,5-Dibromo-N-((2S)-2-(4-fluorophenyl)-4-{3-[(3R)-3-(hydroxymethyl)morpholin-4-yl]azetidin-1-yl}butyl)-N-methylbenzamide; and
3-Cyano-N-((2S)-2-(4-fluorophenyl)-4-{3-[(3R)-3-(hydroxymethyl)morpholin-4-yl]azetidin-1-yl}butyl)-N-methyl-5,6,7,8-tetrahydronaphthalene-1-carboxamide.

15. (canceled)

16. A method for the treatment of a functional gastrointestinal disorder which comprises administering to a patient in need thereof a therapeutically effective amount of a compound according to any one of claims 1-3.

17. A method for the treatment of IBS which comprises administering to a patient in need thereof a therapeutically effective amount of a compound according to any one of claims 1-3.

18. A method for the treatment of functional dyspepsia which comprises administering to a patient in need thereof a therapeutically effective amount of a compound according to any one of claims 1-3.

19. A pharmaceutical formulation comprising a compound according to claim 1 as active ingredient and a pharmaceutically acceptable carrier or diluent.

20. A process for preparing a compound of formula (I) comprising the steps of
a) reacting a compound of the formula (III) with a compound of the formula (IV): 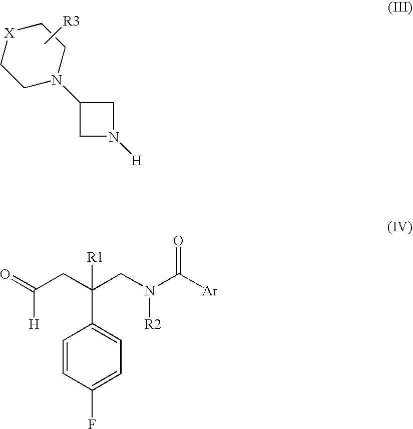
wherein R1-R3 and Ar are as hereinbefore defined; and the conditions are such that reductive alkylation of the compounds of the formula (III) forms an N—C bond between the nitrogen atom of the azetidine group of the compounds of formula (III) and the carbon atom of the aldehyde group of the compounds of formula (IV); or
b) reacting a compound of the formula (III) with a compound of the formula (V): 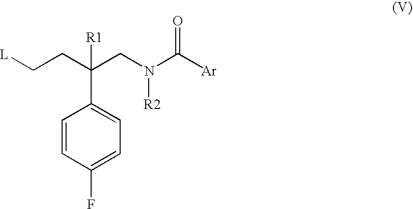
wherein R1-R3 and Ar are as hereinbefore defined; and L is a group such that alkylation of the compounds of the formula (III) forms an N—C bond between the nitrogen atom of the azetidine group of the compounds of formula (III) and the carbon atom of the compounds of formula (V) that is adjacent to the L group; or
c) reacting a compound of the formula (VI) with a compound of the formula (VII): 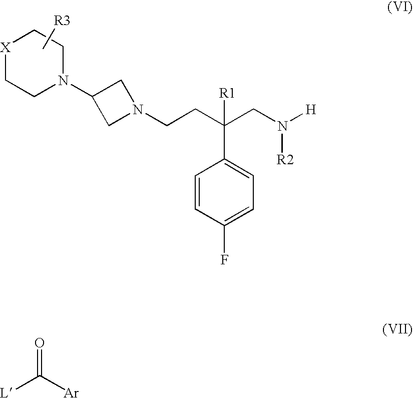
wherein R1-R3 and Ar are as hereinbefore defined; and L′ is a leaving group;
wherein any other functional group is protected, if necessary, and:
i) removing any protecting groups;
ii) optionally oxidizing any oxidizable atoms;
iii) optionally forming a pharmaceutically acceptable salt.

21. A compound selected from
tert-Butyl 4-{1-[(3S)-4-[(3,5-dibromobenzoyl)(methyl)amino]-3-(4-fluorophenyl)butyl]azetidin-3-yl}-3-(2-hydroxyethyl)piperazine-1-carboxylate;
tert-Butyl 4-{1-[(3S)-4-[[(3-cyano-5,6,7,8-tetrahydronaphthalen-1-yl)carbonyl](methyl)amino]-3-(4-fluorophenyl)butyl]azetidin-3-yl}-3-(2-hydroxyethyl)piperazine-1-carboxylate;
tert-Butyl 4-{1-[(3S)-4-[(3-cyano-1-naphthoyl)(methyl)amino]-3-(4-fluorophenyl)butyl]azetidin-3-yl}-3-(hydroxymethyl)piperazine-1-carboxylate;
tert-Butyl 4-{1-[(3S)-4-[(3,5-dibromobenzoyl)(methyl)amino]-3-(4-fluorophenyl)butyl]azetidin-3-yl}-3-(hydroxymethyl)piperazine-1-carboxylate;
3-Bromo-N-[(2S)-2-(4-fluorophenyl)-4-oxobutyl]-N-methyl-5-(trifluoromethyl)benzamide;
2-[(3R)-4-Azetidin-3-ylmorpholin-3-yl]ethanol; and
[(3R)-4-Azetidin-3-ylmorpholin-3-yl]methanol.

22. The method according to claim 16, wherein the compound is the (S)-enantiomer.

23. The method according to claim 17, wherein the compound is the (S)-enantiomer.

24. The method according to claim 18, wherein the compound is the (S)-enantiomer.

25. A method for antagonizing tachykinin action at the NK (neurokinin) receptors in a patient, which comprises administering to the patient a therapeutically effective amount of a compound according to any one of claims 1-3.

26. The method according to claim 25, wherein the compound is the (S)-enantiomer.
